(19) United States
(12) Patent Application Publication
(10) Pub. No.: US 2020/0397319 A1
(43) Pub. Date: Dec. 24, 2020

(54) WEARABLE APPARATUS AND METHOD OF ADJUSTING THE SAME

(71) Applicant: BOE TECHNOLOGY GROUP CO., LTD., BEIJING (CN)

(72) Inventors: Zifeng WANG, Beijing (CN); Yan REN, Beijing (CN); Lei CAO, Beijing (CN); Kai ZHAO, Beijing (CN); Junmin SUN, Beijing (CN); Changlin LENG, Beijing (CN)

(21) Appl. No.: 16/491,076

(22) PCT Filed: Mar. 25, 2019
(86) PCT No.: PCT/CN2019/079480

§ 371 Date: Sep. 4, 2019

(30) Foreign Application Priority Data
May 10, 2018 (CN) 201810445044.3

(51) Int. Cl. A61B 5/021; A61B 5/00

(52) U.S. Cl. CPC A61B 5/02141; A61B 5/681

(57) ABSTRACT

A wearable apparatus and a method of adjusting the same, wherein the wearable apparatus comprises: a determining circuit configured to determine whether the wearable apparatus has been switched to a preset operating mode; an adjusting circuit configured to adjust the wearable apparatus to satisfy an operating requirement of a current operating mode when the determining circuit determines that the wearable apparatus has been switched to the preset operating mode.

### CROSS REFERENCE TO RELATED APPLICATIONS

This application claims priority from Chinese patent application No. 201810445044.3, filed May 10, 2018, the disclosure of which is incorporated herein in its entirety by reference.

### TECHNICAL FIELD

The disclosure relates to a wearable apparatus and a method of adjusting the wearable apparatus.

### BACKGROUND

With the application development of electronic technology, the detecting and the monitoring of health information by the wearable apparatus become a daily application; for example, a blood pressure detection is achieved by a worn wristwatch.

Currently, when the blood pressure is detected through a wristwatch, the blood pressure detection is mainly achieved through the obtained photoplethysmography pulse wave signal (PPG) and the Electro cardio signal (ECG); the main process comprises the following steps: detecting and obtaining the electro cardio potential difference and the pulse of a detected user through three electrodes and a photoelectric tube; calculating and obtaining the blood pressure of the detected user through the detected electro cardio potential difference and the pulse; wherein when the above detection is performed, two electrodes are required to be in close contact with one hand of the detected user, and the remaining one electrode is required to be in close contact with the other hand of the detected user.

### SUMMARY

The following is a summary of the subject matter that is described in detail in this disclosure. This summary is not intended to limit the scope of the claims.

The embodiment of the disclosure provides a wearable apparatus and a method of adjusting the same, which can improve the operating stability of the wearable apparatus.

The embodiment of the disclosure provides a wearable apparatus comprising: a determining circuit and an adjusting circuit; wherein,

the determining circuit is configured to determine whether the wearable apparatus has been switched to a preset operating mode;

the adjusting circuit is configured to adjust the wearable apparatus to satisfy an operating requirement of a current operating mode when the determining circuit determines that the wearable apparatus has been switched to the preset operating mode.

According to an embodiment of the disclosure, wherein the preset operating mode comprises a blood pressure detection mode.

According to an embodiment of the disclosure, wherein the wearable apparatus comprises a wristwatch provided with a first electrode, a second electrode and a third electrode for a blood pressure detection; and

wherein the first electrode is provided on a back of a dial of the wrist watch; the second electrode is provided on a surface of a watchband of the wristwatch, which is in contact with a skin.

According to an embodiment of the disclosure, wherein the adjusting circuit is configured to:

adjust a tightness of the watchband through a preset adjusting device when the determining circuit determines that the wearable apparatus has been switched to the blood pressure detection mode;

stop the adjusting of the tightness of the watchband by the preset adjusting device when a first sensor determines that the adjusting of the tightness of the watchband satisfies a requirement for the blood pressure detection and contacts between the first electrode and the second electrode and the skin satisfy the requirement for the blood pressure detection.

According to embodiments of the disclosure, wherein the wearable apparatus further comprises a detecting circuit and a switching circuit; wherein,

the detecting circuit is configured to detect and determine, through a second sensor, whether a triggering operation satisfying a preset condition is received;

the switching circuit is configured to switch the wearable apparatus to a preset operating mode when the detecting circuit detects the triggering operation satisfying the preset condition.

According to another aspect, there is provided a method of adjusting a wearable apparatus, comprising:

determining whether the wearable apparatus has been switched to a preset operating mode;

adjusting the wearable apparatus to satisfy an operating requirement of a current operating mode when it is determined that the wearable apparatus has been switched to the preset operating mode.

According to embodiments of the disclosure, wherein the preset operating mode comprises a blood pressure detection mode.

According to embodiments of the disclosure, wherein the wearable apparatus comprises a wristwatch provided with a first electrode, a second electrode and a third electrode for a blood pressure detection; and

wherein the first electrode is provided on a back of a dial of the wristwatch; the second electrode is provided on a surface of a watchband of the wristwatch, which is in contact with a skin.

According to embodiments of the disclosure, wherein adjusting the wearable apparatus comprises:

adjusting a tightness of the watchband through a preset adjusting device when it is determined that the wearable apparatus has been switched to the blood pressure detection mode;

stopping the adjusting of the tightness of the watchband by the preset adjusting device when it is determined through a preset first sensor that the adjusting of the tightness of the watchband satisfies a requirement for the blood pressure detection and contacts between the first electrode and the second electrode and the skin satisfy the requirement for the blood pressure detection.

According to embodiments of the disclosure, wherein before determining whether the wearable apparatus has been switched to the preset operating mode, the method further comprises:

detecting and determining, through a second sensor, whether a triggering operation satisfying a preset condition is received;

switching the wearable apparatus to the preset operating mode upon detecting the triggering operation satisfying the preset condition.

### BRIEF DESCRIPTION OF THE DRAWINGS

The accompanying drawings are included to provide a further understanding of the disclosed embodiments and are incorporated in and constitute a part of this specification, are intended to explain the solutions of the disclosure together with the embodiments of the disclosure and not to limit the scope of the disclosure.

FIG. 1 is a schematic view showing the composition of a wristwatch according to the related art;

FIG. 2 is a schematic view showing the composition of a wearable apparatus according to embodiments of the disclosure;

FIG. 3 is a flowchart of a method of adjusting the wearable apparatus according to the embodiments of the disclosure;

FIG. 4 is a schematic view of a display of conventional information for an exemplary wristwatch according to the application examples of the present disclosure;

FIG. 5 is a schematic view of information displayed during a blood pressure detection by an exemplary wristwatch according to the application examples of the present disclosure;

FIG. 6 is a schematic view of the distribution of various electrodes for blood pressure detection according to the application examples of the present disclosure;

FIG. 7 is a schematic view of the position of the wristwatch during daily wearing;

FIG. 8 is a schematic view of the angle range of movement according to embodiments of the present disclosure;

FIG. 9 is a schematic view of the motion of rotating a wrist according to the application examples of the present disclosure;

FIG. 10 is a schematic diagram of the waveform monitored during the rotating of the wrist according to embodiments of the present disclosure;

FIG. 11 is a schematic view of adjusting a tightness of a watchband according to the application examples of the present disclosure;

FIG. 12 is a schematic view showing a composition structure of a wristwatch according to the application examples of the present disclosure.

### DETAILED DESCRIPTION

In order to make the objects, technical solutions and advantages of the present disclosure more apparent, embodiments of the present disclosure will be described in detail below with reference to the accompanying drawings. It should be noted that the embodiments and the features in the embodiments in the present disclosure can be arbitrarily combined with each other without conflict.

The steps illustrated in the flow charts of the drawings may be performed in a set of computer-executable instructions in a computer system. Also, while a logical order is shown in the flow charts, in some cases, the steps as shown or described may be performed in an order different than that as shown or described herein.

FIG. 1 is a schematic view showing the composition of a wristwatch in the related art. As shown in FIG. 1, two electrodes are provided on the back of the wristwatch, and one electrode is provided on the face of the wristwatch. When the two electrodes on the back of the wristwatch are in close contact with one hand of a detected user and the other hand is in close contact with the electrode on the face of the wristwatch, the blood pressure detection can be achieved.

In order to achieve accurate blood pressure detection by the wristwatch, the detected user needs to keep a fixed posture within a certain time period, otherwise, the blood pressure detection is easy to fail. In order to make the two electrodes on the back tightly contact with the hand on which the wristwatch is worn, the other hand of the user is required to apply pressure to the wristwatch, and the operation experience of the detected user is poor. Once the blood pressure detection fails, the detected user needs to perform detection operation again, the detection efficiency is low, the use experience of the user is reduced, and the stability of the blood pressure detection process is poor. In addition to the blood pressure detection, the wristwatch, in most of the time, is configured to display the customized information including time, weather or the like. In the related art, the mode switching between the blood pressure detection and the display of the customized information is mainly realized through preset physical keys, so that the problem of inconvenience in user operation exists.

As compared with the related art, the technical solution of the disclosure comprises: determining whether the wearable apparatus has been switched to a preset operating mode; adjusting the wearable apparatus to satisfy the operating requirement of the current operating mode when it is determined that the wearable apparatus has been switched to the preset operating mode. The embodiments of the disclosure adjust the wearable apparatus through an adjusting device and have improved the operating stability of the preset operating mode.

Additional features and advantages of the disclosure will be set forth in the description which follows, and in part will be apparent from the description, or may be learned by practice of the disclosure. The objectives and other advantages of the disclosure may be realized and attained by the structure particularly pointed out in the description and claims hereof as well as the accompanying drawings.

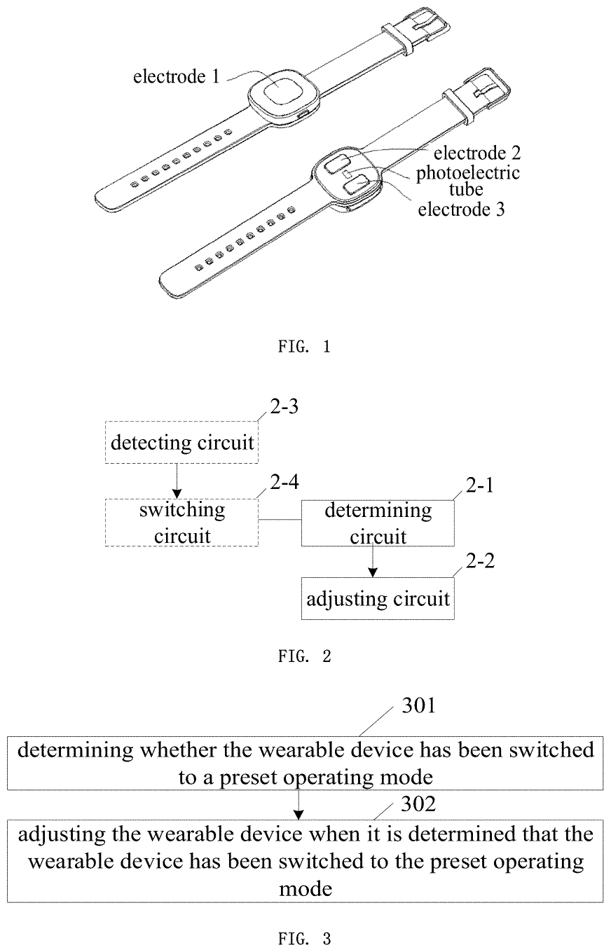

FIG. 2 is a schematic view showing the composition of a wearable apparatus according to embodiments of the disclosure. As shown in FIG. 2, the wearable apparatus comprises: a determining circuit 201 and an adjusting circuit 202; wherein,

the determining circuit 201 is configured to determine whether the wearable apparatus has been switched to a preset operating mode;

the adjusting circuit 202 is configured to adjust the wearable apparatus to satisfy the operating requirement of the current operating mode when the determining circuit determines that the wearable apparatus has been switched to the preset operating mode.

It should be noted that the determining circuit 201 and the adjusting circuit 202 in the embodiments of the present disclosure may be implemented by the processing by an existing controller inside the wearable apparatus; or may be implemented by the processing by a newly configured controller.

According to embodiments of the present disclosure, the preset operating mode in the embodiments of the present disclosure comprises a blood pressure detection mode. Here, the blood pressure detection mode refers to a mode in which the wearable apparatus is switched to a function of detecting blood pressure.

According to embodiments of the present disclosure, the wearable apparatus in the embodiments of the present disclosure may comprise a wristwatch provided with a first electrode, a second electrode and a third electrode for performing blood pressure detection.

According to embodiments of the present disclosure, in the wristwatch as described above according to the embodiments of the present disclosure, the first electrode is provided on the back of the dial of the wristwatch; the second electrode is arranged on a surface of the watchband of the wristwatch, which is contacted with the skin of a user.

According to the embodiments of the disclosure, the third electrode in the embodiments of the disclosure may be provided on the dial of the wristwatch and is configured to realize the contact with the skin of the detected user, which satisfies the blood pressure detection. For example, it can be provided on the outer side of the dial for the contact with the skin of the other hand except the hand of the detected user on which the wristwatch is worn.

It should be noted that the first electrode, the second electrode and the third electrode are the existing electrodes configured to detect the electro cardio potential difference during the blood pressure detection process, and the circuit composition thereof is the same as that of the related art. According to the embodiments of the disclosure, the distribution of the first electrode, the second electrode and the third electrode may be adjusted through the analysis, and the adjusted electrode distribution is combined with the adjusting device, so that the user operation during the blood pressure detection can be simplified.

When the blood pressure detection is carried out according to the embodiments of the disclosure, the wearable apparatus is adjusted through the preset adjusting device, and the stability of the blood pressure detection is improved. For example, according to the embodiments of the present disclosure, the first electrode and the second electrode may be caused to perform skin contact with the first hand by the adjusting device, and the user does not need to press the wristwatch by using the other hand, so that the operation of blood pressure detection is simplified, the failure rate caused by operation is reduced, and the efficiency of the blood pressure detection is improved.

According to the embodiments of the present disclosure, the adjusting unit 2-2 in the embodiments of the present disclosure may, for example, be configured to:

adjust the tightness of the watchband through a preset adjusting device when it is determined that the wearable apparatus has been switched to the blood pressure detection mode;

stop the adjusting of the tightness of the watchband by the preset adjusting device when it is determined through a first sensor that the adjusting of the tightness of the watchband satisfies the requirement for the blood pressure detection and the contacts between the first electrode and the second electrode and the skin of the detected user satisfy the requirement for the blood pressure detection. Here, the first sensor may be an infrared sensor for determining a distance from a wrist of the user on which the wristwatch is worn to the first electrode and/or the second electrode.

According to the embodiments of the disclosure, the wearable apparatus in the embodiments of the disclosure further comprises a detecting circuit 2-3 and a switching circuit 2-4; wherein,

the detecting circuit 2-3 is configured to detect and determine, through a second sensor, whether a triggering operation satisfying a preset condition is received. Here, the second sensor may include one independent sensor, or may include a group of sensors that can detect a plurality of pieces of information.

The switching circuit 2-4 is configured to switch the wearable apparatus to the preset operating mode when the detection circuit detects the triggering operation satisfying the preset condition.

According to the embodiments of the present disclosure, the triggering operation satisfying the preset condition in the embodiments of the present disclosure comprises:

the wearable apparatus is in a preset angle range of movement during a first preset time period with reference to a preset horizontal plane; and/or

a motion with a preset movement pattern occurs on the wearable apparatus during a second preset time period. Here, the triggering operation of the preset movement pattern may be set in combination with the habit of wearing the wristwatch by the detected user.

As compared with the related art, the technical solution of the disclosure comprises: determining whether the wearable apparatus has been switched to a preset operating mode; adjusting the wearable apparatus to satisfy the operating requirement of the current operating mode when it is determined that the wearable apparatus has been switched to the preset operating mode. According to the embodiments of the disclosure, the wearable apparatus is adjusted through the adjusting device, so that the operating stability of the preset operating mode is improved.

FIG. 3 is a flowchart of a method of adjusting a wearable apparatus according to the embodiments of the disclosure.

step 301: determining whether the wearable apparatus has been switched to a preset operating mode;

step 302: adjusting the wearable apparatus to satisfy the operating requirement of the current operating mode when it is determined that the wearable apparatus has been switched to the preset operating mode.

According to the embodiments of the present disclosure, the preset operating mode in the embodiments of the present disclosure comprises a blood pressure detection mode. Here, the blood pressure detection mode refers to a mode in which the wearable apparatus is switched to a function of detecting a blood pressure.

According to the embodiments of the present disclosure, the wearable apparatus in the embodiments of the present disclosure comprises a wristwatch provided with a first electrode, a second electrode and a third electrode for the blood pressure detection;

wherein the first electrode is provided on the back of the dial of the wristwatch; the second electrode is provided on a surface of the watchband of the wristwatch, which is contacted with the skin of a user.

According to the embodiments of the disclosure, the third electrode in the embodiments of the disclosure may be provided on the dial of the wristwatch and is configured to realize the contact with the skin of the detected user, which satisfies the blood pressure detection. For example, it can be provided on the outer side of the dial of the wristwatch for the contact with the skin of the other hand except the hand of the detected user on which the wristwatch is worn.

According to the embodiments of the present disclosure, adjusting the wearable apparatus comprises:

adjusting the tightness of the watchband through a preset adjusting device when it is determined that the wearable apparatus has been switched to the blood pressure detection mode;

stopping the adjusting of the tightness of the watchband by the preset adjusting device when it is determined through a preset first sensor that the adjusting of the tightness of the watchband satisfies a requirement for the blood pressure detection and the contacts between the first electrode and the second electrode and the skin satisfy the requirement for the blood pressure detection.

According to the embodiments of the disclosure, before determining whether the wearable apparatus has been switched to the preset operating mode, the method further comprises:

detecting and determining, through a preset sensor, whether a triggering operation satisfying a preset condition is received;

switching the wearable apparatus to the preset operating mode upon detecting the triggering operation satisfying the preset condition.

According to the embodiments of the present disclosure, the triggering operation satisfying the preset condition comprises:

the wearable apparatus is in a preset angle range of movement during a first preset time period with reference to a preset horizontal plane; and/or

a motion with a preset movement pattern occurs on the wearable apparatus during a second preset time period.

The embodiments of the present disclosure further provide a terminal comprises: a memory and a processor; wherein,

the processor is configured to execute program instructions in the memory;

the program instructions are read by the processor to perform the following operations: determining whether the wearable apparatus has been switched to a preset operating mode;

adjusting the wearable apparatus through a preset adjusting device to satisfy the operating requirement of the current operating mode when it is determined that the wearable apparatus has been switched to the preset operating mode.

The following provides a clear and detailed description of the method of the embodiments of the present disclosure by way of application examples, which are only intended for the purpose of illustrating the present disclosure and are not intended to limit the scope of the present disclosure.

Application Examples

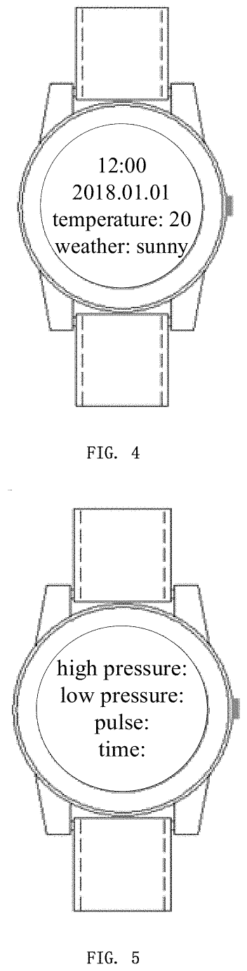

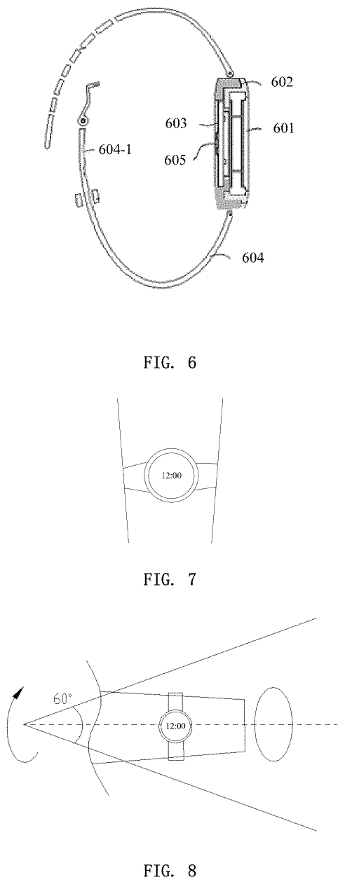

The application examples are described by taking a wearable apparatus as a wristwatch and a preset operating mode as a blood pressure detection mode as an example. The wristwatch related to the application examples may be configured to display time, weather and other customized information in addition to the blood pressure detection. The application examples do not make any adjustments with respect to the display of information except the blood pressure detection with reference to the related art. FIG. 4 is a schematic view of a display of conventional information for an exemplary wristwatch according to the application examples of the present disclosure. As shown in FIG. 4, the customized information such as time, temperature, weather or the like is displayed on the display interface of the wristwatch. FIG. 5 is a schematic view of information displayed during a blood pressure detection by an exemplary wristwatch according to the application examples of the present disclosure. As shown in FIG. 5, data related to the blood pressure detection is displayed on the display interface of the wristwatch during the blood pressure detection. FIG. 6 is a schematic view of the distribution of various electrodes for blood pressure detection according to the application examples of the present disclosure. As shown in FIG. 6, the wristwatch comprises a display interface 601, a dial 602, a back 603 (the back case of the wristwatch) and a watchband 604. Based on the above structure, a first electrode is provided on the back 603 of the wristwatch; the second electrode is connected to the watchband 604, the end 604-1 of which is adapted to contact the skin of the detected user during the blood pressure detection; the dial 602 is a third electrode for detecting the blood pressure; the three electrodes are connected to a circuit board in the wristwatch in different ways to realize the detection of the electro cardio potential difference; a photoelectric tube 605 is provided in the back 603 of the wristwatch and is configured to detect the pulse information, and the blood pressure information may be obtained by combining the electro cardio potential difference detected by the electrodes and the pulse information detected by the photoelectric tube; the result of the blood pressure detection is displayed on the display interface 601.

When the blood pressure detection is carried out, the two hands of the detected user are required to cooperate for a certain time period. For example, for the wristwatch as shown in FIG. 6, when the adjusting device is not provided, the user needs to apply pressure to the wristwatch when the user uses one hand to contact the third electrode, so that the first electrode and the second electrode are in contact with the skin of the hand on which the wristwatch is worn, which satisfies the requirements. Thereby, the continuous electro cardio potential difference detection is performed. When the time is long, the detected user is easy to fatigue, and the detection process is easy to fail. According to the application examples, when it is determined that the trigger signal of switching to the blood pressure detection mode is detected, the preset adjusting device is configured to adjust the tightness of the watchband 604 of the wristwatch. Thus, without the pressing operation, the first electrode and the second electrode can be in contact with the skin of the hand on which the wristwatch is worn, which satisfies the detection requirement. Therefore, the blood pressure detection process is more convenient. After obtaining the information of the blood pressure detection, the application examples can automatically adjust the watchband 604 to the previous state and control the wristwatch to display the customized information.

The process of switching to the blood pressure detection mode and the process of adjusting the tightness of the watchband are respectively illustrated as follows.

The application examples detect and determine whether the wristwatch is switched to the blood pressure detection mode by a preset sensor. For example, a trigger operation satisfying a preset condition may be set with reference to an operation habit of the detected user. When the preset sensor detects the triggering operation, the triggering information is generated, and the wristwatch is switched to the blood pressure detection mode according to the generated trigger information.

FIG. 7 is a schematic view of the position of the wristwatch during daily wearing. As shown in FIG. 7, the wristwatch is generally at the lower end of the wrist due to the weight of the wristwatch itself, which is the loosest state of the watchband. When the user lifts a hand and needs to view the wristwatch, the user can sometimes support the wristwatch by the other hand to be convenient to view. Alternatively, the user can rotate the wrist with one hand, and when the arm is lifted, the wristwatch is also moved upwards, so that the watchband is clamped, and the wristwatch is fixed at a better position to be viewed. Based on the above habit, the embodiments of the disclosure set the following triggering operation with the following preset movement pattern to switch the wristwatch to the blood pressure detection mode. The triggering operation satisfying the preset condition according to the application examples comprises:

the wristwatch is in a preset angle range of movement during a preset time period with reference to a preset horizontal plane; and/or

a motion with a preset movement pattern occurs during a second preset time period.

The wearable apparatus being in the preset angle range of movement during the preset time period comprises: setting a horizontal plane according to the wearing angle of the wristwatch when the detected user views the wristwatch; and the angle at which the wristwatch performs a rotation movement during a certain time period is +/−30 degrees based on the set horizontal plane. FIG. 8 is a schematic view of the angle range of movement according to embodiments of the present disclosure. As shown in FIG. 8, all the range of 60 degrees as shown in the figure is the exemplary angle range of movement according to the application examples. The above angle set by the application examples can facilitate the user to view the result of the blood pressure detection on the wristwatch. The application examples can carry out the monitoring of the angle of movement of the wristwatch through a gyroscope sensor. According to the application examples, the motion with the preset movement pattern occurring during the second preset time period may comprise a simple touch operation, a click operation or the like. For example, the setting of the motion with the preset movement pattern based on the operation habit of the detected user may comprise:

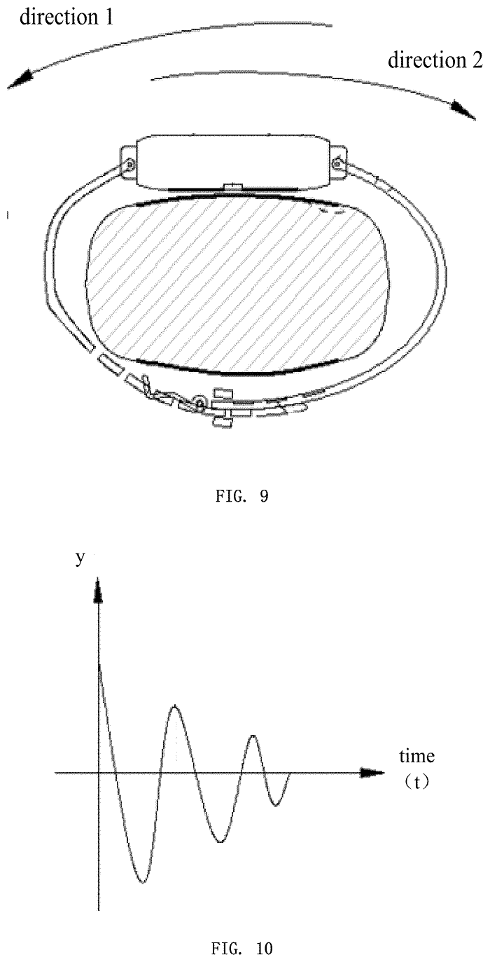

the wrist of the detected user is rotated for more than the preset number of times during the second preset time period; the preset number of times may be 3. FIG. 9 is a schematic view of the motion of rotating a wrist according to the application examples of the present disclosure. As shown in FIG. 9, when the detected user rotates the wrist, the wristwatch will repeatedly move in the directions 1 and 2 as shown in the figure. According to the application examples, the damping vibration curve can be detected by the photoelectric tube. When the wrist of the detected user is rotated, the waveform as shown in FIG. 10 can be monitored by the infrared light emitted by the photoelectric tube, and the number of wave peaks is equivalent to the number of times of rotating the wrist.

According to the application examples, after the trigger signal is generated, the photoelectric tube emits green light to perform the blood pressure detection.

It should be noted that the above-mentioned exemplary operations may not be performed simultaneously, and allow a certain time difference. For example, two conditions may occur one after another within 3 seconds.

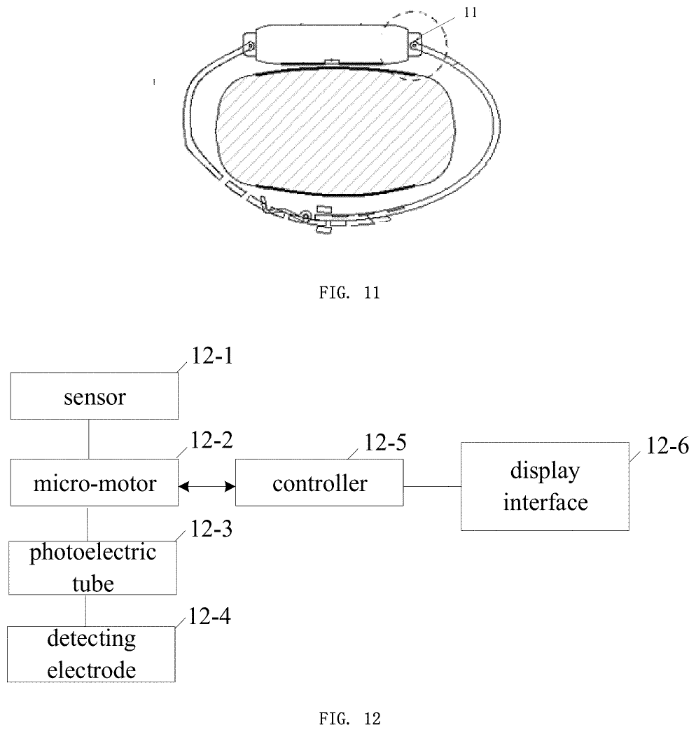

FIG. 11 is a schematic view of adjusting a tightness of a watchband according to the application examples of the present disclosure. As shown in FIG. 11, in order to make the first electrode and the second electrode have good contact with the skin, an adjusting device 11 may be provided at the end of the watchband according to the application examples. The adjusting device 11 may comprise: means configured to adjust the tightness of the watchband by taking a micro-motor as power. The realization principle thereof can be designed and realized by referring to the principle of the shrinkage of the mechanical arm of the micro robot. Namely, when it is switched to the blood pressure detection mode, the micro motor is enabled to control the tightness of the watchband, so that the first electrode and the second electrode can fit the skin better. When the blood pressure detection is completed, according to the application examples, the micro motor is controlled to restore the watchband to the state before the blood pressure detection, so that the tightness of the wristwatch worn by the detected user satisfies the requirement of comfort level. FIG. 12 is a schematic view showing a composition structure of a wristwatch according to the application examples of the present disclosure. As shown in FIG. 12, the wristwatch comprises a sensor 12-1, a micro motor 12-2, a photoelectric tube 12-3, a detection electrode 12-4 for detecting the electro cardio potential difference, a controller 12-5 and a display interface 12-6. The controller is configured to process the data collected by the sensor, control the operations of the micro motor and the photoelectric tube, and process the collected blood pressure detection data.

It will be appreciated by those of ordinary skill in the art that all or a part of the steps of the above-described methods may be completed by the related hardware (for example a processor) which is instructed by a program that may be stored on a computer-readable storage medium, such as a read-only memory, magnetic or optical disk or the like. Alternatively, all or part of the steps of the above-described embodiments may be implemented by using one or more integrated circuits. Accordingly, each module/unit in the above embodiments may be implemented in the form of hardware (for example, its corresponding function may be implemented by an integrated circuit), or may be implemented in the form of a software functional module (for example, its corresponding function may be implemented by a processor executing programs/instructions stored in a memory). The present disclosure is not limited to any specific form of combination of hardware and software.

Although the embodiments disclosed in the present disclosure are described above, the descriptions are only adopted for facilitating understanding of the present disclosure, and are not intended to limit the present disclosure. Any person skilled in the art to which this disclosure pertains may make any changes or modifications in form and detail without departing from the spirit and scope of this disclosure, and the scope of protection of the disclosure is to be determined by that of the appended claims.

1. A wearable apparatus comprising: a determining circuit and an adjusting circuit; wherein,
the determining circuit is configured to determine whether the wearable apparatus is switched to a preset operating mode;
the adjusting circuit is configured to adjust the wearable apparatus to satisfy an operating requirement of a current operating mode when the determining circuit determines that the wearable apparatus has been switched to the preset operating mode.

2. The wearable apparatus of claim 1, wherein the preset operating mode comprises a blood pressure detection mode.

3. The wearable apparatus of claim 2, wherein the wearable apparatus comprises a wristwatch provided with a first electrode, a second electrode and a third electrode for a blood pressure detection; and
wherein the first electrode is provided on a back of a dial of the wristwatch; the second electrode is provided on a surface of a watchband of the wristwatch, which is in contact with a skin.

4. The wearable apparatus of claim 3, wherein the adjusting circuit is configured to:
adjust a tightness of the watchband through a preset adjusting device when the determining circuit determines that the wearable apparatus has been switched to the blood pressure detection mode;
stop the adjusting of the tightness of the watchband by the adjusting device when a first sensor determines that the adjusting of the tightness of the watchband satisfies a requirement for the blood pressure detection and contacts between the first electrode and the second electrode and the skin satisfy the requirement for the blood pressure detection.

5. The wearable apparatus of claim 4, wherein the adjusting device comprises means configured to adjust the tightness of the watchband by taking a micro-motor as power.

6. The wearable apparatus of claim 1, wherein the wearable apparatus further comprises a detecting circuit and a switching circuit; wherein,
the detecting circuit is configured to detect and determine, through a second sensor, whether a triggering operation satisfying a preset condition is received;
the switching circuit is used for switching the wearable apparatus to a preset operating mode when the detecting circuit detects the triggering operation satisfying the preset condition.

7. The wearable apparatus of claim 6, wherein the triggering operation satisfying the preset condition comprises:
the wearable apparatus is in a preset angle range of movement during a first preset time period with reference to a preset horizontal plane; and/or
a motion with a preset movement pattern occurs on the wearable apparatus during a second preset time period.

8. The wearable apparatus of claim 7, wherein the wearable apparatus being in the preset angle range of movement during the first preset time period comprises:
setting a horizontal plane according to a wearing angle of the wearable apparatus when the wearable apparatus is viewed;
an angle at which the wearable apparatus performs a rotational movement during the first preset time period is +/−30 degrees based on the set horizontal plane.

9. The wearable apparatus of claim 7, wherein the motion with the preset movement pattern occurring on the wearable apparatus during the second preset time period comprises:
a number of times that the wearable apparatus rotates during the second preset time period is greater than a preset number of times.

10. A method of adjusting a wearable apparatus, comprising:
determining whether the wearable apparatus has been switched to a preset operating mode;
adjusting the wearable apparatus to satisfy an operating requirement of a current operating mode when it is determined that the wearable apparatus has been switched to the preset operating mode.

11. The method of claim 10, wherein the preset operating mode comprises a blood pressure detection mode.

12. The method of claim 11, wherein the wearable apparatus comprises a wristwatch provided with a first electrode, a second electrode and a third electrode for a blood pressure detection; and
wherein the first electrode is provided on a back of a dial of the wristwatch; the second electrode is provided on a surface of a watchband of the wristwatch, which is in contact with a skin.

13. The method of claim 12, wherein adjusting the wearable apparatus comprises:
adjusting a tightness of the watchband through a preset adjusting device when it is determined that the wearable apparatus has been switched to the blood pressure detection mode;
stopping the adjusting of the tightness of the watchband by the preset adjusting device when it is determined, through a preset first sensor, that the adjusting of the tightness of the watchband satisfies a requirement for the blood pressure detection and contacts between the first electrode and the second electrode and the skin satisfy the requirement for the blood pressure detection.

14. The wearable apparatus of claim 13, wherein the adjusting device comprises means configured to adjust the tightness of the watchband by taking a micro-motor as power.

15. The method of claim 10, wherein before determining whether the wearable apparatus has been switched to the preset operating mode, the method further comprises:
detecting and determining, through a second sensor, whether a triggering operation satisfying a preset condition is received;
switching the wearable apparatus to the preset operating mode upon detecting the triggering operation satisfying the preset condition.

16. The method of claim 15, wherein the triggering operation satisfying the preset condition comprises:
the wearable apparatus is in a preset angle range of movement during a first preset time period with reference to a preset horizontal plane; and/or
a motion with a preset movement pattern occurs on the wearable apparatus during a second preset time period.

17. The method of claim 16, wherein the wearable apparatus being in the preset angle range of movement during the first preset time period comprises:
setting a horizontal plane according to a wearing angle of the wearable apparatus when the wearable apparatus is viewed;
an angle at which the wearable apparatus performs a rotational movement during the first preset time period is +/−30 degrees based on the set horizontal plane.

18. The method of claim 16, wherein the motion with the preset movement pattern occurring on the wearable apparatus during the second preset time period comprises:
a number of times that the wearable apparatus rotates during the second preset time period is greater than a preset number of times.

19. A terminal, comprising:
a memory; and
a processor;
wherein the processor is configured to execute program instructions in the memory;
the program instructions, when read by the processor, perform the method of claim 10.
